(19) United States
(12) Patent Application Publication
(10) Pub. No.: US 2011/0306526 A1
(43) Pub. Date: Dec. 15, 2011

(54) LUBRICATING OIL BASED ON POLYOL ESTERS

(75) Inventors: Laurent Germanaud, Heyrieux (FR); Carine Alfoss, Pessac (FR); Patrick Turello, Craponne (FR); Elise Bataille, Vlissingen (NL)

(73) Assignee: TOTAL RAFFINAGE MARKETING, Puteaux (FR)

(21) Appl. No.: 13/132,875

(22) PCT Filed: Dec. 7, 2009
(86) PCT No.: PCT/IB2009/055553

§ 371 Date: Aug. 29, 2011

(30) Foreign Application Priority Data
Dec. 5, 2008 (FR) 08/06825

(51) Int. Cl. C10M 169/04; C07C 59/40

(52) U.S. Cl. 508/110; 554/122

(57) ABSTRACT

The present invention relates to oils based on polyol esters, which may be obtained from renewable resources and used as lubricant bases or lubrication additives, notably in four-stroke engine oils, oils for hydraulics or transmissions, as well as industrial lubricants.

### TECHNICAL FIELD

The present invention relates to oils based on polyol esters, which may be obtained from renewable resources, and may be used as lubricating bases or lubrication additives, notably in four-stroke engine oils, oils for hydraulics or transmissions, as well as industrial lubricants.

### TECHNICAL BACKGROUND/PRIOR ART

The oils used as lubricating bases in engines or various vehicle components, or in industry, are typically hydrocarbon oils stemming from petroleum cuts.

Oils of vegetable origin are a renewable alternative to these products. They contain in majority esters of glycerol or other polyols and of natural fatty acids. However, the poor cold properties and the low resistance to oxidation of these products limit the use thereof, notably in motor oil formulations. This for example is the case of rapeseed oils and oleic sun flower oils.

Natural fatty acid esters, liquid at room temperature, are unsaturated compounds and therefore sensitive to oxidation. Moreover, saturated natural esters of fatty acids such as lauric, myristic, palmitic or stearic acids are themselves solid at room temperature which makes them unsuitable for use as a lubricating base.

Therefore there exists a need for having compounds of renewable origin having oxidation resistance properties and cold viscosity allowing them to be used in lubricating compositions for vehicles, in particular in combustion engines or for industrial uses.

### SUMMARY OF THE INVENTION

The present invention proposes to solve this problem by providing oils comprising one or more polyol esters, so-called <<mixed>> esters, since, in the synthesis of these compounds, at least one alcohol function of each polyol has been esterified by a natural fatty acid and at least one alcohol function of this same polyol has been esterified by a synthetic fatty acid.

Synthetic fatty acids are typically short chain saturated acids (typically including less than 12 carbon atoms) and natural fatty acids are typically long chain unsaturated acids (typically including at least 14 carbon atoms).

Advantageously, the synthetic fatty acids used for producing the oils according to the present invention may themselves have been obtained from renewable resources, such as for example from heptanoic acid obtained by thermal cracking of castor oil, or from C₈-C₁₀ fatty acid cuts, stemming from the refining and distillation of natural oils such as for example copra oil.

The invention therefore relates to oil comprising at least one tetraester fitting the general formula (I):

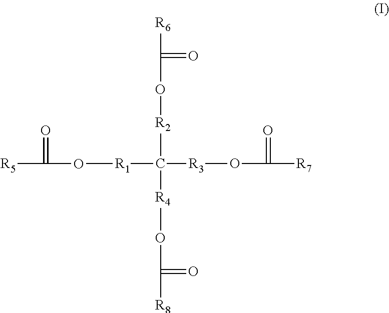

wherein:
the groups R₁, R₂, R₃, R₄ are aliphatic chains including from 1 to 10 carbon atoms;
the groups R₅, R₆, R₇, R₈ are either short paraffinic chains including from 6 to 11 carbon atoms, or long olefinic chains including from 13 to 21 carbon atoms;
at least one of the groups R₅, R₆, R₇, R₈ is a short paraffinic chain including from 6 to 11 carbon atoms and at least one of the groups R₅, R₆, R₇, or R₈ is a long olefinic chain including from 13 to 21 carbon atoms;

wherein,

the ratio between the number of moles of long fatty acids comprising from 14 to 22 carbon atoms and the number of moles of short fatty acids comprising from 7 to 12 carbon atoms, is comprised between 0.3 and 2.5, the ratio being determined on the composition of fatty acid methyl esters obtained from said oil by applying the NF ISO 5509 and NF ISO 5508 standards,

and wherein,

said oil comprises at least 15% by weight, preferentially at least 18% by weight of tetraester(s) of formula (I) wherein 2 of the groups R₅, R₆, R₇, R₈ are short paraffinic chains including from 6 to 11 carbon atoms, and 2 of the groups R₅, R₆, R₇, R₈ are long olefinic chains including from 13 to 21 carbon atoms.

Preferably, R₁, R₂, R₃, R₄ are aliphatic chains including from 1 to 4 carbon atoms.

Preferably, the long fatty acid methyl esters comprising from 14 to 22 carbon atoms are in majority mono-unsaturated, in the fatty acid methyl ester composition, obtained from said oil by applying the NF ISO 5509 and NF ISO 5508 standards, of said oil.

Preferably, the oil comprises at least 30% by weight, preferentially 35% by weight of tetraesters of formula (I), wherein at least two of the groups R₅, R₆, R₇, R₈ are long olefinic chains including from 13 to 21 carbon atoms and/or of a tetraester fitting the general formula ((II)

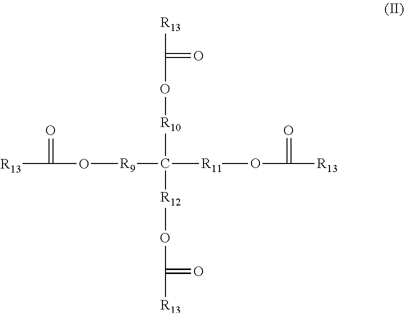

wherein R₉, R₁₀, R₁₁, R₁₂ are aliphatic chains including from 1 to 10 carbon atoms, preferentially from 1 to 4 carbon atoms, and R₁₃ is a long olefinic chain including from 13 to 21 carbon atoms.

Preferably, the oil comprises at most 10%, preferentially at most 7% by weight of tetraesters of formula (II).

Preferably, the oil comprises at most 25% by weight of tetraester of formula (I) wherein 3 of the groups R₅, R₆, R₇, R₈ are long olefinic chains including from 13 to 21 carbon atoms.

Preferably, the oil comprises at least 85% by weight of total or partial ester(s) obtained by reaction of one or more polyols of formula (III)

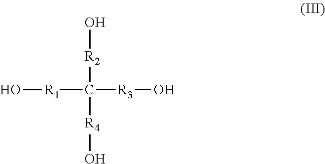

wherein R₁, R₂, R₃, R₄ are aliphatic chains including from 1 to 10 carbon atoms, preferentially from 1 to 4 carbon atoms, with one or more long unsaturated fatty acids comprising from 14 to 22 carbon atoms and/or one or more short saturated fatty acids comprising from 7 to 12 carbon atoms.

Preferably, the oil comprises at least 30% by weight of tetraesters of formula (I) including from 40 to 70 carbon atoms and at least 15% by weight, preferentially at least 20% by weight of tetraesters of formula (I) including from 45 to 60 carbon atoms.

Preferably, the oil has a hydroxyl number, measured according to the NF T60-231 standard, of less than 10 mg of KOH/g.

Preferably, the oil has an acid number, measured according to the NF ISO 660 standard, of less than 1 mg KOH/g.

Preferably, the oil has an iodine number, measured according to the NF ISO 3961 standard, of less than 50, preferentially less than 40, and even more preferentially less than 30 g I₂/100 g.

Preferably, the ratio between the number of moles of long fatty acids comprising from 14 to 22 carbon atoms and the number of moles of short fatty acids comprising from 7 to 12 carbon atoms, is comprised between 1.5 and 2.5, preferentially between 1.6 and 2, the ratio being determined on the composition of fatty acid methyl esters obtained from said oil by applying the NF ISO 5509 and NF ISO 5508 standards.

Preferably, the ratio between the number of moles of long fatty acids comprising from 14 to 22 carbon atoms and the number of moles of short fatty acids comprising from 7 to 12 carbon atoms, is comprised between 0.4 and 1.1, preferentially between 0.42 and 1, the ratio being determined on the composition of fatty acid methyl esters obtained from said oil by applying the NF ISO 5509 and NF ISO 5508 standards.

The object of the present invention is also lubricating compositions containing said oils. In particular it relates to lubricating compositions for four-stroke engines containing said oils and to any type of base oil and additives adapted to this use.

Preferably, the lubricating composition comprises from 10 to 99%, or from 10 to 70%, or from 10 to 40%, or further from 10 to 50%, or from 15 to 30%, even more preferentially from 15 to 25% of an oil as defined above.

Preferably, the lubricating composition further comprises:
From 0 to 70%, or further from 5 to 70%, or from 30 to 70% of one or more base oils selected from mineral oils of the group III and/or synthetic oils of the groups IV, V and VI
From 0 to 30%, or from 2 to 30%, preferentially from 5 to 20% of one or more polymers improving VI, preferentially selected from polymers and copolymers of methacrylates, olefins, styrene or dienes,
From 0.2 to 10%, preferentially from 0.5 to 5%, of one or more anti-oxidant additives, preferentially of the amine and/or phenol type,
From 0.01 to 5% of one or more additives depressing the pour point, preferentially selected from polymers and copolymers of methacrylates.

Preferably, the lubricating composition comprises from 30 to 70% of one or more base oils of the group IV, with a kinematic viscosity at 100° C. comprised between 4 and 8 cSt.

Preferably, the lubricating composition has a kinematic viscosity of 100° C. comprised between 5.6 and 9.3 cSt. (grade 20).

Preferably, the lubricating composition has a kinematic viscosity at 100° C. comprised between 9.3 and 12.5 cSt. (grade 30).

Preferably, the lubricating composition has a viscosity index greater than 160, preferentially greater than 175.

The present invention also relates to the use of these oils based on mixed esters or mixtures of mixed esters as a base oil or friction modifier in lubricating compositions, notably a lubricant for engines, hydraulics, transmissions, and industrial lubricants. It relates to the use of such oils as a single lubricant base for engines, hydraulics and transmissions of vehicles of public works or farm vehicles or further as a lubricant for four-stroke engines, preferentially for engines of lightweight or heavy duty motor vehicles, preferably for a gasoline or diesel engine.

Finally, the present invention relates to a method for producing oils based on mixed esters according to the invention.

The method for producing oil according to the invention comprises:

i) a first step for transesterification of a polyol of formula (III):

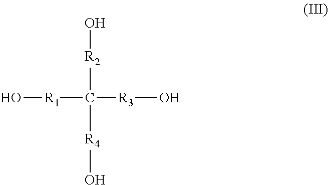

wherein the groups R₁ to R₄ are aliphatic chains including from 1 to 10 carbon atoms, preferentially from 1 to 4 carbon atoms, by one or more saturated short fatty acid methyl esters including from 7 to 12 carbon atoms

in the presence of a homogeneous or heterogeneous transesterification basic catalyst, preferentially selected from sodium methylate, potassium hydroxide, sodium hydroxide, manganese oxide or zinc oxide,

preferentially under a nitrogen flow, preferentially of the order of 30 mL/minute at atmospheric pressure,

preferentially in an initial alcohol/saturated short fatty acid methylester molar ratio comprised between 1/5 and 1.2.5.

This first step comprises the following steps:

i.1: introducing at a temperature of the order of 20 to 25° C., into the reaction mixture formed by the polyol and the saturated short fatty acid methyl ester(s), an amount of catalyst preferentially accounting for between 1 and 2% by mass of the amount of saturated short fatty acid methyl esters,

i.2: raising the temperature of the reaction mixture up to a temperature above 150° C., preferentially comprised between 160 and 180° C.,

i.3: preferably continuously drawing off the methanol produced by the nitrogen flow and condensing the latter,

i.4: maintaining at a temperature above 150° C., preferably comprised between 160 and 180° C., the reaction mixture until the reaction stops, preferably materialized by the stopping of the formation of condensates in the nitrogen flow.

Said first transesterification step (i) results in a reaction product consisting of partial polyol esters,

(ii) A second step for transesterification of one or more reaction products obtained in the first step (i), by one or more long unsaturated fatty acid methyl esters comprising from 14 to 22 carbon atoms, and preferably including a single unsaturation.

This second step is carried out in the presence of a homogeneous or heterogeneous transesterification basic catalyst, preferentially selected from sodium methylate, potassium hydroxide, sodium hydroxide, manganese oxide or zinc oxide, preferentially identical with the one of the first step (i),

preferably in the presence of an anti-foam agent, for example dimethyl polysiloxane (DMS), at a content of about 10 ppm in the reaction medium,

preferably in an average vacuum of the order of 30 millibars.

This second step comprises the following steps:

ii.1: measuring, according to the NFT 60-231 standard, the hydroxyl number of the starting medium formed by a determined amount of one or more products from a first step (i), and calculating the number of non-esterified polyol hydroxyl moles, n OH, present in said medium,

ii.2: introducing into said medium at a temperature of the order of 20 to 25° C., N moles of long unsaturated fatty acid methylester(s), in an N/nOH molar ratio comprised between 0.8 and 1.2, preferentially equal to 1,

ii.3: introducing into said medium, at a temperature of the order of 20 to 25° C., an amount of catalyst representing between 0.5 and 1.5% by mass, preferentially of the order of 0.75% by mass of the amount of long unsaturated fatty acid methyl esters introduced in step ii.2,

ii.4: optionally introducing, into said medium at a temperature of the order of 20 to 25° C., an amount of anti-foam agent representing about 10 ppm of the total reaction mixture,

ii.5: raising the temperature of the thereby formed reaction mixture up to a temperature above 150° C., preferentially comprised between 160 and 170° C.

ii.6: maintaining at this temperature the reaction medium for a period of more than 3 hours.

Preferably, the method further comprises a third step for neutralization of the unreacted hydroxyl groups by acetic anhydride.

Preferably, the mixture of unsaturated long fatty acid methyl esters comprising from 14 to 22 carbon atoms used in step (i) for transesterifying the polyol includes at least 85%, preferentially at least 90% by weight, even more preferentially at least 95% by weight of mono-unsaturated fatty chain methyl esters, said percentage being determined by NF ISO05508.

Preferably, the mono-unsaturated methyl esters comprise from 16 to 22 carbon atoms, preferentially 18 carbon atoms.

Preferably, the polyols are selected from pentaerythritol and neopentylglycol. .

### DETAILED DESCRIPTION

The object of the present invention is oils comprising at least one tetraester fitting the general formula (I):

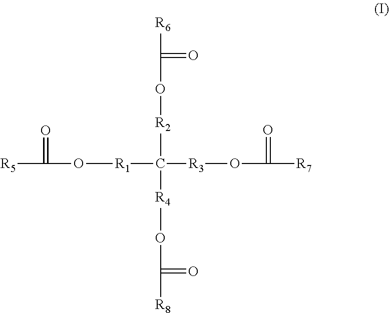

wherein:
the groups R₁, R2, R3, R4 are aliphatic chains including from 1 to 10 carbon atoms
the groups R₅, R₆, R₇, Rs are either short paraffinic chains including from 6 to 11 carbon atoms, or long olefinic chains including from 13 to 21 carbon atoms
at least one of the groups R5, R6, R7, R₈ is a short paraffinic chain including from 6 to 11 carbon atoms and at least one of the groups R₅, R₆, R₇, or R₈ is a long olefinic chain including from 13 to 21 carbon atoms,

wherein,

the ratio between the number of moles of long fatty acids comprising from 14 to 22 carbon atoms and the number of moles of short fatty acids comprising from 7 to 12 carbon atoms, is comprised between 0.3 and 2.5, preferentially comprised between 0.4 and 2, the ratio being determined on the composition of fatty acid methyl esters obtained from said oil by applying the NF ISO 5509 and NF ISO 5508 standards,

and wherein

said oils comprise at least 15% by weight, preferentially at least 18%, even more preferentially at least 20% by weight of tetraester(s) of formula (I) wherein 2 of the groups R₅, R₆, R₇, R₈ are short paraffinic chains including from 6 to 11 carbon atoms, and 2 of the groups R₅, R₆, R₇, R₈ are long olefinic chains including from 13 to 21 carbon atoms.

The groups R₁, R₂, R₃, R₄ are preferentially aliphatic chains including from 1 to 4 carbon atoms.

Preferentially, the long fatty acid methyl esters comprising from 14 to 22 carbon atoms are in majority mono-unsaturated, in the composition of fatty acid methyl esters, determined according to the NF ISO 5509 and NF ISO 5508 standards, of said oil.

The unsaturated long fatty acids unlike their solid saturated homologs at room temperature, have physico-chemical properties allowing the oils which contain them, to be used in lubricating compositions. However, limiting the content of di-, tri-, or poly-unsaturated long fatty acids imparts to said oils better resistance to oxidation.

Preferably, the oils according to the invention contain at least 30% by weight, preferentially 35%, even more preferentially at least 40% by weight of tetraesters of formula (I), wherein at least two of the groups R₅, R₆, R₇, R₈ are long olefinic chains including from 13 to 21 carbon atoms and/or of a tetraester fitting the general formula (II)

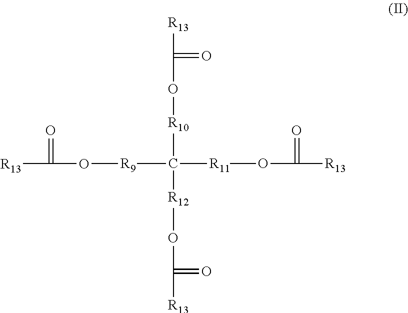

wherein R₉, R₁₀, R₁₁, R₁₂ are aliphatic chains including from 1 to 10 carbon atoms, preferentially from 1 to 4 carbon atoms, and R₁₃ is a long olefinic chain including from 13 to 21 carbon atoms.

Indeed, a minimum content of tetraesters of this type imparts sufficiently high viscosity in order to be able to use the oils containing them as a lubricating composition, notably for the applications more particularly targeted by the present invention, i.e. industrial lubricants and automobile lubricants, in particular for engines, hydraulics and transmissions.

According to an embodiment, the oils according to the invention contain at most 10%, preferentially at most 9%, preferentially at most 7%, preferentially at most 6%, even more preferentially at most 5% by weight of tetraesters of formula (II).

Indeed, this type of ester, if it allows a sufficient viscosity to be guaranteed, however includes at least 4 unsaturations. A too high content of this type of esters may lead to low resistance to oxidation, which may be a penalty for using them in lubricating compositions, notably in engine lubricants.

For the same reasons, the oils according to the invention preferentially contain at most 25%, or further at most 20% or at most 15% by weight of tetraesters of formula (I) wherein 3 of the groups R₅, R₆, R₇, R₈ are long olefinic chains including from 13 to 21 carbon atoms.

The oils according to the invention preferentially contain at least 85%, or further at least 95% by weight of total or partial esters obtained by reaction of one or more polyols of formula (III)

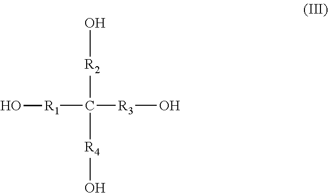

wherein R₁, R₂, R₃, R₄ are aliphatic chains including from 1 to 10 carbon atoms, preferentially from 1 to 4 carbon atoms, with one or more long unsaturated fatty acids comprising from 14 to 22 carbon atoms and/or of short saturated fatty acids comprising from 7 to 12 carbon atoms.

The presence in a too large amount of non-esterified polyols and more generally of non-esterified hydroxyl functions in the oils according to the invention may actually have a negative impact on their use in lubricating compositions. In particular a strong increase in the viscosity induced by the formation of hydrogen bonds between the non-esterified hydroxyl functions may be observed, which would make them unsuitable for use in lubricating compositions.

The mass percentages of the different esters and tetraesters of polyols present in the oils according to the invention are determined from their GPC (gas phase chromatography) analysis.

Preferably, the oils according to the invention comprise at least 30% by weight of tetraesters of formula (I) including from 40 to 70 carbon atoms and at least 15% by weight, preferentially at least 20% by weight of tetraesters of formula (I) including from 45 to 60 carbon atoms.

The mass percentage of tetraesters having a given number of carbon atoms is determined by GPC (gas phase chromatography) analysis of the oils according to the invention, according to the method described in the examples hereafter.

According to a preferred embodiment, the oils according to the invention have a hydroxyl number, measured according to the NFT60-231 standard of less than 10 mg of KOH/g, the hydroxyl number allows quantification of the non-esterified hydroxyl functions in the oils.

A limited content of such free hydroxyl functions correlated with a low hydroxyl number, gives the possibility of having oils with adequate viscosimetric properties for use in lubricating compositions. The formation of hydrogen bonds between the molecules, as mentioned above is minimized and which leads to very strong increases in viscosity.

Preferentially, the oils according to the invention have an acid number, measured according to the NF ISO 660 standard, of less than 1 mg KOH/g. The acid number in mg of KOH/gram of product, allows quantification of the unreacted fatty acids (the higher the number, the more there are unreacted fatty acids).

A low acid number therefore also reveals a limited content of unreacted hydroxyl and therefore gives the possibility of obtaining oils having viscosimetric properties more adapted to use in lubricating compositions.

Preferentially, the oils according to the invention have an iodine number, measured according to the NF ISO 3961 standard, of less than 50, preferentially less than 40, even more preferentially less than 30, or less than 15, or less than 10 grams of I₂ for 100 grams of oil.

The iodine number is related to the presence of unsaturations and therefore to the sensitivity to oxidation. The lower the number and the less there are unsaturations, better therefore is the resistance to oxidation. Oils having a low iodine number will therefore be able to be used in applications where the oxidation resistance parameter is important, for example in engine lubricant compositions.

According to an alternative, the oils according to the invention have a ratio, between the number of moles of long fatty acids comprising from 14 to 22 carbon atoms and the number of moles of short fatty acids comprising from 7 to 12 carbon atoms, comprised between 1.50 and 2.50, preferentially between 1.60 and 2.00, even more preferentially between 1.61 and 1.90. This ratio is determined on the composition of fatty acid methyl esters obtained from said oil by applying the NF ISO 5509 and NF ISO 5508 standards.

The oils according to this alternative may be used for example as lubricating bases in industrial lubricant applications.

These oils have the viscosity required for an application in the field of industrial lubricants, as well as good cold properties. However, their resistance to oxidation is limited. Their viscosity at 100° C. according to ASTMD 445 is preferentially comprised between 4 and 10 mm²/s, preferentially between 6 and 9 mm²/s, even more preferentially between 8 and 9 mm²/s

Their dynamic viscosity at −25° C., measured according to the ASTM D5293 standard is typically less than 4,300, preferentially less than 3,500 mPa·s.

According to another alternative, the oils according to the invention have a ratio, obtained from their composition of fatty acid methyl esters according to the NF ISO 5509 and NF ISO 5508 standards, between the number of moles of long fatty acids comprising from 14 to 22 carbon atoms and the number of moles of short fatty acids comprising from 7 to 12 carbon atoms, which is comprised between 0.4 and 1.49, preferentially between 0.4 and 1.20, even more preferentially between 0.42 and 1.10, or further between 0.42 and 1.00.

Thus, the oils having these long fatty acids/short fatty acid molar ratio values have the thermo-oxidative properties required for an application as a lubricating base in lubricating compositions for engines. Some examples hereafter detail these properties in a high temperature oxidation ICOT test and in an MCT test, which quantifies the tendency of forming deposits on a hot surface.

The viscosity of said oils is also adapted to their use, notably for formulating grade 20 or 30 oils according to the SAE (Society of Automotive Engineers) classification.

Preferentially they have a kinematic viscosity of 100° C., measured according to the ASTM D 445, comprised between 4 and 8 mm²/s, preferentially between 4 and 6.5 mm²/s.

Their viscosity index, according to the ASTM D2270 is preferentially greater than or equal to 150, preferentially greater than or equal to 155.

Their cold properties may, in a suitable formulation (notably with pour point depressing additives and a suitable polymer improving VI), allow the formulation of multigrade 5W or even 0W multigrade motor oils, notably 5W30 and 0W30 oils according to the SAE classification.

The object of the present invention is also lubricating compositions comprising an oil according to the invention as described above.

More particularly, its object is lubricating compositions comprising oils according to the invention which have a ratio between the number of moles of long fatty acids comprising from 14 to 22 carbon atoms and the number of moles of short fatty acids comprising from 7 to 12 carbon atoms, comprised between 0.4 and 1.49, preferentially between 0.4 and 1.20, and even more preferentially between 0.42 and 1.10, or further between 0.42 and 1.00. This ratio is determined on the composition of fatty acid methyl esters obtained from said oil by applying the NF ISO 5509 and NF ISO 5508 standards.

Said lubricating compositions preferentially comprise from 10 to 99%, or from 10 to 70%, or further from 10 to 40%, from 10 to 50%, or from 15 to 30%, or even more preferentially 15 to 25% of such oils.

They may further comprise:
from 0 to70%, or further from 5 to 70%, or from 30 to 70% of one or more base oils selected from mineral oils of the group III and/or synthetic oils of groups IV, V et VI
from 0 to 30%, or from 2 to 30%, preferentially from 5 to 20% of one or more polymers improving the VI, preferentially selected from polymers and copolymers of methacrylates, olefins, styrene or dienes.
from 0.2 to 10%, preferentially from 0.5 to 5%, of one or more anti-oxidant additives, preferentially of the amine and/or phenol type,
from 0.01 to 5% of one or more pour point depressing additives, preferentially selected from polymers and copolymers of methacrylates.

According to a particularly preferred embodiment, said compositions comprise from 30 to 70% of one or more base oils of the group IV, with kinematic viscosity of the 100° C. comprised between 4 and 8 mm²/s

According to an embodiment, these compositions have a kinematic viscosity at 100° C. comprised between 5.6 and 9.3 mm²/s, which corresponds to grade 20 oils according to the SAE classification.

According to another embodiment, these lubricating compositions have a kinematic viscosity at 100° C. comprised between 9.3 and 12.5 mm²/s, which corresponds to grade 30 oils according to the SAE classification. Their viscosity index is preferentially greater than 160, even more preferentially greater than 175.

The object of the present invention is also the use of the oils described above as a friction modifier additive and as a lubricating base in lubricating compositions.

In particular the object thereof is the use of oils according to the invention, having a ratio between the number of moles of long fatty acids comprising from 14 to 22 carbon atoms and the number of moles of short fatty acids comprising from 7 to 12 carbon atoms, comprised between 1.50 and 2.50, preferentially between 1.60 and 2.00, even more preferentially between 1.61 and 1.90, as a lubricant base, for a hydraulic lubricant, lubricant for transmissions, and for industrial lubricants. This ratio is determined on the fatty acid methyl ester composition obtained from said oil by applying the NF ISO 5509 and NF ISO 5508 standards.

The object is also in particular the use of oils according to the invention, having a ratio between the number of moles of long fatty acids comprising from 14 to 22 carbon atoms and the number of moles of short fatty acids comprising from 7 to 12 carbon atoms, comprised between 0.4 and 1.49, preferentially between 0.4 and 1.20, even more preferentially between 0.42 and 1.10, or further between 0.42 and 1.00, as a lubricant base for a lubricant for engines, hydraulics, transmissions and for industrial lubricants. This ratio is determined on the fatty acid methyl ester composition obtained from said oil by applying the NF ISO 5509 and NF ISO 5508 standards.

Preferentially, the object is the use of the latter oils as a lubricant base for formulating a single lubricant which may be used both for engines, hydraulics and transmissions of public work vehicles or farm vehicles.

The present invention also relates to the use of lubricating compositions as described above as a lubricant for four-stroke engines, preferentially for engines of lightweight or heavy duty motor vehicles.

Method for Preparing the Oils

Finally, the object of the present invention is also a method for producing oils as defined above, comprising:

i) a first step for transesterification of a polyol of formula (III):

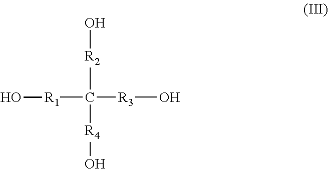

wherein the groups R₁ to R₄ are aliphatic chains including from 1 to 10 carbon atoms, preferentially from 1 to 4 carbon atoms, by one or more saturated short fatty acid methyl esters including from 7 to 12 carbon atoms,

in the presence of a homogeneous or heterogeneous transesterification basic catalyst, preferentially selected from sodium methylate, potassium hydroxide, sodium hydroxide, manganese oxide or zinc oxide,

under nitrogen flow, preferentially of the order of 30 mL/minute at atmospheric pressure,

in an initial polyol/saturated fatty acid methyl esters molar ratio comprised between 1/5 and 1/2.5,

comprising the steps of:

i.1: introducing at a temperature of the order of 20 to 25° C., into the reaction medium formed by the polyol and the saturated short fatty acid methyl ester(s), an amount of catalyst representing between 1 and 2%, typically 1.4% by mass of the amount of saturated short fatty acid methyl esters,

i.2: raising the temperature of the reaction mixture up to a temperature above 150° C., preferably comprised between 160 and 180° C., preferentially of the order of 170° C.

i.3: preferably, continuously drawing off the methanol produced by the nitrogen flow and condensing the latter,

i.4: preferably maintaining the reaction mixture at a temperature comprised between 160 and 180° C., preferentially of the order of 170° C., until the reaction stops, materialized by the stopping of the formation of condensates in the nitrogen flow.

Said first transesterification step (i) resulting in a reaction product consisting of partial polyol esters,

(ii) a second step for transesterifying one or more reaction products obtained in a first step (i), by one or more long unsaturated fatty acid methyl esters comprising from 14 to 22 carbon atoms, and preferably including a single unsaturation, in the presence of a homogeneous or heterogeneous transesterification basic catalyst, preferentially selected from sodium methalate, potassium hydroxide, sodium hydroxide, manganese oxide or zinc oxide, preferentially identical with that of the first step (i),

preferably in the presence of an anti-foam agent for example dimethyl polysiloxane (DMS), at a content of about 10 ppm in the reaction medium,

preferably in an average vacuum of the order of 30 millibars,

comprising the steps of:

ii.1: measuring according to the NFT 60-231 standard, the hydroxyl number of the starting medium formed by a determined amount of one or more products from a first step (i), and calculating the number of non-esterified hydroxyl moles of polyol, nOH, present in said medium,

ii.2: introducing into said medium at a temperature of the order of 20 to 25° C., N moles of the long unsaturated fatty acid methyl ester(s) in a molar ratio N/nOH comprised between 0.8 and 1.2, preferentially between 0.9 and 1.1, preferentially equal to 1,

ii.3: introducing into said medium, at a temperature of the order of 20 to 25° C., an amount of catalyst representing between 0.5 and 1.5% by mass, preferentially of the order of 0.75% by mass, of the amount of long unsaturated fatty acid methyl esters introduced in step ii.2,

ii.4: preferably introducing into said medium at a temperature of the order of 20 to 25° C., an amount of anti-foam agent accounting for about 10 ppm of the total reaction mixture,

ii.5. preferably raising the temperature of the thereby formed reaction mixture up to a temperature comprised between 160 and 170° C., preferentially of the order of 165° C., and then

ii.6 maintaining at this temperature the reaction medium for a period comprised between 3 and 7 hours.

According to an embodiment, the method according to the invention further includes a third step for neutralizing the unreacted hydroxyl groups with acetic anhydride.

Preferentially, in the method according to the invention, the mixture of unsaturated long fatty acid methyl esters comprising from 14 to 22 carbon atoms used in step (i) for transesterifying the polyol includes at least 85%, preferentially at least 90% by weight, even more preferentially at least 95% by weight of mono-unsaturated methyl esters, said percentage being determined by NF ISO 5508.

Preferentially, the mixture of unsaturated long fatty acid methyl esters used in step (i) for transesterifying the polyol includes at least 80%, preferentially at least 85%, preferentially at least 90% by weight, even more preferentially at least 95% by weight of mono-unsaturated methyl esters comprising from 16 to 22 carbon atoms, preferentially 18 carbon atoms, said percentage being determined by NF ISO 5508.

Preferentially, the polyols are selected from pentaerythritol and neopentylglycol.

The object of the present invention is also products which may be obtained by the methods described above.

Characterization of the Oils.

The oils according to the invention are mainly characterized from two types of analysis.

1. Their composition of fatty acid methyl esters, and more particularly by the ratio between the number of long chain fatty acid methyl esters and the number of moles of short chain fatty acid methyl esters which is inferred therefrom (which is equivalent to the ratio between the number of moles of long fatty acids comprising from 14 to 22 carbon atoms and the number of moles of short fatty acids comprising from 7 to 12 carbon atoms).

This ratio is determined by the composition of fatty acid methyl esters obtained from said oil by applying the NF ISO 5509 and NF ISO 5508 standards as follows: The composition of fatty acid methyl esters of an oil is made in two steps:
fatty acid methyl esters are prepared from said oil according to the EN ISO 5509 standard,
the obtained mixture of methyl esters is then analyzed by gas phase chromatography according to the EN ISO 5508 standard.

The mass percentage of the different fatty acid methyl esters in the oil is then obtained. By knowing the molar mass of these different methyl esters, it is therefore possible to calculate the molar ratios between these different esters present in the analyzed oil.

The oils according to the invention contain esters of polyols, esterified by two types of fatty acids:

The so-called “long” fatty acids are defined as fatty acids comprising from 14 to 22 carbon atoms. These long fatty acids are in principle unsaturated, but the mixtures used in practice for synthesizing the oils may contain minority amounts of saturated substances (cf. Example 1 hereafter). For calculating the characteristic molar ratio of the oils according to the invention, methyl esters of all the fatty acids comprising from 14 to 22 carbon atoms will be taken into account.

The so-called “short” fatty acids are defined as fatty acids comprising from 7 to 12 carbon atoms. These short fatty acids are in principle exclusively saturated. For calculating the characteristic molar ratio of the oils according to the invention, methyl esters of all the fatty acids comprising from 7 to 12 carbon atoms will however be taken into account.

It is also from the composition obtained via NF ISO 5509/5508 that it is possible to determine whether the methyl esters of long fatty acids comprising from 14 to 22 carbon atoms are in majority mono-unsaturated in the oils according to the invention.

This is the case when one or more mono-unsaturated methyl esters are the most abundant species, in moles of mono-unsaturated methyl ester(s)/moles of chromatographable species, according to said composition in accordance with NF ISO 5509/5508.

2. Their mass composition of polyol esters is obtained by GPC analysis (gas phase chromatography), and by the average carbon number of the polyol esters which they contain, which is also obtained from GPC analysis.

The method used, which is detailed in the Example 1 hereafter, again takes up the characteristics of the IUPAC 2.323 method used for determining triglycerides. The separation of the different species is accomplished per increasing carbon number. According to the IUPAC method, the column is calibrated by having a mixture of reference triglycerides with a known composition, pass through it. The polyol esters of the oils according to the invention flow out at the same retention time as the one for triglycerides with a same carbon number.

With this method it is possible to distinguish polyol tetraesters including:
Four “long” acid chains (designated hereafter by 4C₁₈ esters)
Three long acid chains and one short acid chain (designated hereafter by 3C₁₈C₈)
Two long acid chains and two short chains (designated hereafter by 2C₁₈2C₈).

The terms of long acid and short acid have the meaning specified above.

So-called “partial” esters i.e. the esters comprising one or more non-esterified OH functions, the tetraesters including three short chains and one long chain (3C₈1C₁₈), the tetraesters including four short chains (4C₈), cannot be separated with this method, because of their too close carbon number.

The results are given as a mass percentage based on the total of the chromatographable species. The latter comprise:
The unreacted reaction products (polyol, C₇-C₁₂ short fatty acid methyl esters, C₁₄-C₂₂ long fatty acid methyl esters,
<<Partial>> esters (for all the products according to the invention, partial esters include the tetraesters with three short chains and one long chain, as well as the tetraesters with four short chains and the esters on which remain one or more free OH functions)
The tetraesters (other than those included in the partial esters).

This method identifies the different species present depending on their carbon number. Therefore this method will be used for calculating the mass percentage of polyol esters including from 40 to 70 carbon atoms, or further from 45 to 60 carbon atoms, in the oils according to the invention.

For this, the mass percentage of the species having retention times comprised between those of the reference triglycerides with 40 and 70 carbon atoms, or with 45 and 60 carbon atoms, will be calculated, based on the total of the chromatographable species.

Lubricating Compositions.

The object of the present invention is also lubricating compositions comprising oils based on esters of polyols according to the present invention, regardless of their application, whether they are for example intended for engine, hydraulic, transmission applications or industrial applications.

More particularly, the present invention relates to lubricating compositions for four-stroke engines, including the oils according to the present invention, and any type of additives or base oils suitable for their use.

In particular, the present invention relates to lubricating compositions for four-stroke engines preferentially comprising from 10 to 99%, or from 10 to 70%, or further from 10 to 40%, from 10 to 50%, or 15 to 30%, still more preferentially 15 to 25% of such oils.

They may further comprise:
from 0 to 70%, or further from 5 to 70%, or from 30 to 70% of one or more base oils selected from mineral oils of group III and/or synthetic oils of groups IV, V and VI
from 0 to 30%, or from 2 to 30%, preferentially from 5 to 20% of one or more polymers improving VI, preferentially selected from polymers and copolymers of methacrylates, olefins, styrene or dienes,
from 0.2 to 10%, preferentially from 0.5 to 5%, of one or more anti-oxidant additives, preferably of the amine and/or phenol type,
from 0.01 to 5% of one or more pour point depressing additives, preferentially selected from polymers and copolymers of methacrylates.

According to a particularly preferred embodiment, said compositions comprise from 30 to 70% of one or more base oils of group IV, with a kinematic viscosity at 100° C. comprised between 4 and 8 mm²/s

According to a still more preferred embodiment, the lubrication compositions for four-stroke engines according to the present invention also comprise:
from 0 to 45% of one or more base oils of group IV, with a viscosity of 6 mm²/s at 100° C.
from 0 to 45% of one or more base oils of group IV, with a kinematic viscosity of 4 mm²/s at 100° C.,
from 5 to 10% of one or more polymers improving VI
from 0.2 to 5% of one or more anti-oxidant additives
from 0.01 to 5% of one or more pour point depressing additives.

Non-limiting examples of additives which may enter the lubricating compositions according to the invention, are given below.

Antioxidant Additives:

These additives delay the degradation of the oils during operation, which may be expressed by the formation of deposits, the presence of sludge, or an increase in the viscosity of the oil. They act as radical inhibitors or hydroperoxide destructors. Among the currently used antioxidants, are found antioxidants of the phenol, amine types. Some of these additives, for example phosphosulfur additives, may be generators of ashes.

Phenol antioxidants may be without any ashes, or else be in the form of neutral or basic metal salts. Typically, these are compounds which contain a sterically hindered hydroxyl group, for example when 2 phenol groups are in the ortho or para position relatively to each other, or when the phenol is substituted with an alkyl group of at least 6 carbon atoms.

Amino compounds are another class of antioxidants which may be used, optionally in combination with phenol compounds. Typical examples are aromatic amines of formula R₈R₉R₁₀N, wherein R₈ is an aliphatic group, or an optionally substituted aromatic group, R₉ is an optionally substituted group, R₁₀ is hydrogen, or an alkyl or aryl group, or a group of formula R₁₁S(O)xR₁₂, wherein R₁₁ is an alkylene, alkenylene, or aralkylene group and x is equal to 0, 1 or 2.

Sulfurized alkyl phenols or their alkaline and earth alkaline metal salts are also used as antioxidants.

Organic boron derivatives such as esters or succinimides may also be used as antioxidants.

Another class of antioxidants are copper compounds soluble in the oil, for example copper thio- or dithio-phosphates, salts of copper and carboxylic acids, copper dithiocarbonates, sulfonates, phenates, acetylacetonates. Copper(I) and (II) salts with succinic acid or anhydride are used.

Additives Lowering the Pour Point

They improve the hot behavior of oils, by slowing down the formation of paraffin crystals.

For example, these are polyalkyl methacrylates, polyacrylates, polymers of esters of fumaric or maleic acid and heavy alcohols, copolymers of different esters of acrylic, methacrylic, fumaric or maleic acid, or further copolymers of esters of fumaric acid and of vinyl esters of fatty acids, copolymers of fumarates, vinyl esters of carboxylic acids, and of alkyl vinyl ethers, or their mixture.

In this category of additives, polyacrylamides, polyalkylphenols, polyalkylnaphthalenes, alkyl polystyrene . . . , condensation products of paraffins or halogenated waxes and of aromatic compounds such as benzene, naphthalene, anthracene, phenols, are notably found.

Polymers Improving Viscosity.

With them it is possible to guarantee good cold resistance and a minimum viscosity at high temperature, notably for formulating multigrade oils. The introduction of these compounds into the lubricating compositions enable them to reach viscosity index (VI) values providing them with good “eco” or fuel-saving properties.

Thus, the lubricated compositions according to the invention have VI values measured according to ASTM D2270, greater than or equal to 160, preferentially greater than 175, preferentially greater than 180.

For example among these compounds improving the viscosity index, mention may be made of polymeric esters, olefinic copolymers (OCP), homopolymers or copolymers of styrene, of butadiene or isoprene polymethacrylates (PMA). Conventionally they are present at levels of the order of 0 to 40%, preferentially from 0.01 to 15% by weight in lubricating compositions for four-stroke engines.

The preferred VI-improving polymers are selected from polymers and copolymers of methacrylates, olefins, styrene or dienes.

Other Additives.

The lubricating compositions for engines according to the invention may moreover contain any types of additives suitable for their use, for example:
anti-wear and extreme-pressure agents protecting the friction surfaces by reaction with the metal surface,
friction modifying additives, forming a protective film adsorbed on the friction surfaces, and among which are for example found fatty amines, fatty alcohols, fatty esters,
dispersants ensuring that insoluble solid contaminants are maintained in suspension and discharged,
either overbased detergents or not, reducing the formation of deposits at the surface of the metal parts by dissolution of the secondary oxidation and combustion products,
anti-rust and anti-corrosion additives
anti-foam additives, . . .

These additives may be individually introduced into the lubricating compositions or in the form of packets of additives or concentrates of additives.

The nature and the proportion of the different base oils and additives in the lubricating compositions according to the present invention will preferentially be adjusted so that said lubricating compositions are of grade 20 or 30 according to the SAE classification, with a kinematic viscosity of 100° C. comprised between 5.6 and 9.3 or comprised between 9.3 and 12.5 cSt, and their high viscosity index, which may be greater than or equal to 160 for oils of grade 20 and greater than or equal to 175 for oils of grade 30.

Even more preferentially, these lubricating compositions are multi-grade oils, for example 5W or 0W, for example of grade 5W30 or 0W30 according to the SAE classification.

Use:

The present invention also relates to the use of an oil according to the invention as a friction modifying additive in lubricating compositions.

The use as a friction modifier utilizes the property which fatty esters have, such as those present in the oils according to the invention, of forming at the surface of the frictional paths, films with which hydrodynamic flow may be maintained under a strong load.

When they are used as a friction modifier, the oils according to the invention are typically incorporated at contents of less than 10% or even less than 5%, typically comprised between 1 and 2%.

The present invention also relates to the use of an oil according to the invention as a lubricant base, alone or mixed with oils of natural, animal or vegetable, mineral origin or synthetic oils.

In particular, the present invention relates to the use of an oil according to the invention as a lubricant base for engines, hydraulics, transmissions, and industrial lubricants.

The use of oil according to the invention as a lubricant base is particularly suitable for open air and leisure applications, such as agricultural machinery, site construction machinery, leisure vehicles, where biodegradability is desired, but the oils according to the present invention may be used in multiple applications, including industrial lubricants.

The oils according to the invention may be used as a single lubricant base for engines, hydraulics and transmissions of vehicles, notably for formulating lubricants which may be used equally in engines, in hydraulics and in the transmission of a same vehicle. This type of single lubricant may in particular be applied to public works' vehicles or farm vehicles.

Method for Producing the Oils:

These oils are typically obtained by transesterification of polyols by short chain synthetic fatty acid methyl esters comprising between 7 and 12 carbon atoms, followed by transesterification by long chain natural fatty acid methyl esters, comprising between 14 and 22 carbon atoms, in the presence of basic transesterification catalysts.

These catalysts may for example be selected from homogeneous catalysts such as sodium methylate, potassium hydroxide, sodium hydroxide, or heterogeneous catalysts such as manganese oxide or zinc oxide.

An additional esterification step in the presence of acetic anhydride may be added in order to neutralize the remaining hydroxyl functions and obtain a better tetraester yield, which improves the physical characteristics of the obtained oils, notably viscosity and pour point.

The operating procedure of this synthesis is detailed in Example 1 hereafter.

Polyols.

The polyols used for obtaining the compounds according to the invention are tetra-alcohols. Preferentially, the tetra-alcohols used for preparing the oils according to the invention fit the formula (III) below wherein R₁, R₂, R₃, R₄ are aliphatic chains including from 1 to 10 carbon atoms, preferentially 1 to 4 carbon atoms.

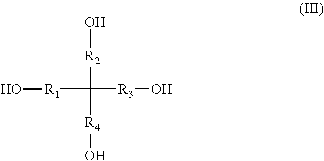

The preferred tetra-alcohols are pentaerythritol (R₁═R₂═R₃═R₄═C₂H₄) and neopentylglycol (R₁═R₂R₃═R₄═CH₂).

The oils according to the present invention have the particularity of containing polyol tetraesters esterified both by unsaturated long fatty acids and saturated short fatty acids.

Unsaturated Long Fatty Acids:

By “long” fatty acids, are meant here fatty acids comprising between 14 and 22 carbon atoms. The saturated long acids are solid at room temperature and therefore unsuitable for use in the synthesis of lubricants. Therefore unsaturated long acids are used here.

In order to impart to the oils according to the invention resistance to oxidation suitable for the targeted uses, notably in engine lubricants, mono-unsaturated long acids will be preferred. Palmitoleic, oleic, eicosenoic, erucic acids, in particular oleic acid, will be preferred.

The benefit of these long acids is that they may stem from natural resources. In order to synthesize the oils according to the invention, unsaturated long fatty acids of natural origin are therefore preferably used. They are present, in the form of their methyl esters, in oils of vegetable or animal origin such as palm, sunflower, rapeseed oil, olive oil, groundnut oil . . . , which may be refined, enriched, genetically modified, . . . so as to increase their content of fatty acids of interest.

In order to achieve the synthesis of the compounds according to the invention, sunflower oil enriched with methyl oleate or rapeseed oil will advantageously be used.

These natural raw materials are mixtures, which also generally contain more or less significant amounts of methyl esters of polyunsaturated fatty acids (linoleic, linolenic acid for example), as well as a few methyl esters of saturated fatty acids (myristic, palmitic, stearic, behenic acid for example).

Saturated Short Fatty Acids:

By “short” fatty acids are meant here fatty acids comprising between 7 and 12 carbon atoms. These saturated acids have the benefit of reinforcing resistance to oxidation of the oils according to the invention without any detrimental effect on their lubricating properties.

Mention will notably be made of caproic, heptanoic, caprylic, pelargonic and capric acids. The fatty acids including 7 and 8 carbon atoms are particularly preferred.

However, unlike the long acid described above, they are not available in nature. Therefore synthetic saturated short fatty acids are used. They may for example be obtained from petroleum cuts. Heptanoic acid obtained by heat-cracking of castor oil may be used advantageously. C₈-C₁₀ cuts may also be advantageously used, mainly lean C₁₀ cuts.

### EXAMPLES

### Example 1

### Synthesis and Characterization of the Oils Based on Mixed Esters

Preparation Method:

Several oils have been prepared by transesterifying in a first step pentaerythritol (PET) by saturated C₈-C₁₀ fatty acid methyl esters (VOME), and then by transesterifying in a second step the resulting product by unsaturated long fatty acid methyl esters (SOME). The resulting oils PET 9-1, PET 12-1, PET 25-3, PET 28-2, PET 29-1 are oils according to the invention.

Raw Materials:

Polyol: pentaerythritol (PET) of formula C(CH₂OH)₄, with 98% purity, marketed by Aldrich (CAS no. 115-77-5, M.W: 136) is used as a tetra-alcohol.

Saturated short fatty acid methyl esters: a mixture of methyl caprate and caprylate marketed by Oleon (VOME), containing 55% by weight of caprylic esters and 40% by weight of capric ester and with an average molar mass of 169 g/mol, is used.

Unsaturated long fatty acid methyl esters: a mixture of oleic sunflower oil methyl esters (SOME), rich in mono-unsaturated methyl oleate is used. Its composition (NF ISO 5509/5508) is given in the table below. Its average molar mass is M=295.5 g/mol.

TABLE 1
composition of the mixture of unsaturated long fatty acid methyl esters.

| Nature of the Methyl | |
|---|---|
| Ester | Composition (%) |
| C 14:0 | 0.0 |
| C 16:0 | 3.8 |
| C 16:1 | 0.1 |
| C 18:0 | 3.2 |
| C 18:1 | 78.6 |
| C 18:2 | 11.0 |
| C 18:3 | 0.7 |
| C 20:0 | 0.3 |
| C 20:1 | 0.4 |
| C 22 | 0.9 |
| C 24 | 0.3 |

Operating Procedure for the First Step:
In a 250 mL reactor equipped with a reflux heating circuit and a Dean-Stark, x eq. of PET are mixed with y eq. of VOME. (x and y are numbers of moles calculated from average molar masses of the reagents). The mixture is placed under a constant flow of N₂, intended to gradually extract the formed methanol, and with stirring (600 rpm).
In certain cases, the reaction mixture is preheated to a temperature of 145° C., in other cases, it is maintained at room temperature (20° C.)
The N₂ bubbling and stirring are stopped in order to introduce into the reaction medium the catalyst MeONa (1.4% based on the initially introduced mass of VOME). Once the addition is finished, N₂ bubbling and stirring are immediately restarted.
The mixture is then brought to the reaction temperature, comprised between 145 and 170° C.
This temperature is maintained until the reaction is completed, ascertained by the stopping of methanol distillate production in the effluent. (The reaction time is the time during which this temperature is maintained).
The distillate (methanol drawn off gradually by the N₂ flow) is recovered and the reaction raw material is analyzed.
No formation of foam is seen.
The majority of the methanol is distilled during the 1^{st} hour of reaction.
The hydroxyl number OHN (mg of KOH/g of product, according to the NF T 60-231 standard) on the final raw product is measured at the end of the reaction in order to evaluate progress in the reaction. The amount of moles of unreacted hydroxyls of the initial polyol, nOH, is calculated. In x grams of final product, there are thus: nOH=x. (NOH/56100) moles of unreacted OH.
The mass y of methyl oleate equivalent to be introduced into the second step is thereby calculated, which corresponds to N moles of methyl oleate equivalent,
N/nOH is set to 1, whence y=M.N=M.x.(OHN/56100), with M the average molar mass M (g/mol) of the methyl oleate mixture (SOME) used for the second transesterification step.

Operating Procedure for the Second Step:
In a 250mL reactor, equipped with a vacuum distillation assembly, x grams of PET esters stemming from the first step are mixed with y grams of SOME dried beforehand at 90° C. in a vacuum of 10 mbars for one hour
The reaction mixture is stirred (600 rpm) and optionally heated: in certain cases, the reaction mixture is preheated to a temperature of 80° C., in other case, it is maintained at room temperature (20° C.). MeONa (catalyst) and DMPS (anti-foam agent, dimethyl polysiloxane) are then introduced, and the medium is placed in a vacuum of 30 mbars.
The reaction medium is then heated up to a reaction temperature comprised between 130° C. (with preheating) and 165° C. (without preheating)
After 2 to 6 hours of maintaining this temperature, the vacuum is cut off, the heating and stirring are stopped. (The reaction time is the time during which this temperature is maintained).
One operates in vacuo in order to reduce the reaction temperature
N2 bubbling was suppressed: indeed, if this step is carried out under a flow of N₂ foaming is observed with the consequence of carrying away the reaction medium out of the reactor, and therefore it is impossible to conduct the reaction to completion.

Neutralization of the Remaining OH (Optional):

Tests were conducted in order to reduce the amount of unreacted hydroxyl functions in the medium. Indeed, free hydroxyl functions have the particularity of forming intermolecular hydrogen bonds, which increases the viscosity of the medium. In order to avoid this phenomenon, the final product may be esterified by an acid or even an acetic anhydride at the end of the reaction.

Treatment

The raw reaction mixture is washed 3 times with salted water, and then 3 times with demineralized water. Centrifugation may be necessary during the 1^{st} washing in order to increase the decantation rate.

The organic phase is dried at 100° C. in a vacuum of 10 mbars in order to remove residual water.

Table 2 below groups the different experimental conditions under which these two (optionally three) successive steps were performed.

TABLE 2
conditions for synthesis of PET esters

| | | | | | | |
|---|---|---|---|---|---|---|
| Table 2 | PET 9-1 | PET 12-1 | PET 15-3 | PET 25-3 | PET28-2 | PET 29-1 |

1^{st} step: transesterification of PET by VOME

| | PET Trans | | PET Trans | | PET Trans | | | | | | | |
|---|---|---|---|---|---|---|---|---|---|---|---|---|
| | C8C10 9 | | C8C10 12 | | C8C10 15 | | | | | | | |
| M PET (g) | 138.7 | | 160.1 | | 45 | | | | | | | |
| n PET/n VOME | 1/2.4 | | 1/2.4 | | 1/3.7 | | 1/3.7 | | 1/2.9 | | 1/4.9 | |
| (molar ratios) | | | | | | | | | | | | |
| % of MeONa catalyst | 1.4% | | 1.4% | | 1.4% | | 1.4% | | 1.4% | | 1.4% | |
| (MeONa masses, based | (m/m | | (m/m | | (m/m | | (m/m | | (m/m | | (m/m | |
| on the introduced mass | VOME) | | VOME) | | VOME) | | VOME) | | VOME) | | VOME) | |
| of VOME) | | | | | | | | | | | | |
| Introduction of the | at 145° | C. | at 145° | C. | at 145° | C. | at 20° | C. | at 20° | C. | at 20° | C. |
| catalyst | | | | | | | | | | | | |
| Reaction temperature | 145 | | 175 | | 175 | | 170 | | 170 | | 170 | |
| (° C.) | | | | | | | | | | | | |
| N₂ Flow rate | ~25 | mL/min | ~25 | mL/min | ~30 | mL/min | ~30 | mL/min | ~30 | mL/min | ~30 | mL/min |

| | | | | | | | | | |
|---|---|---|---|---|---|---|---|---|---|
| Reaction time (hours) | 7 | h | 7 | h | 6 | h | 1 h-2 h | 1 h-2 h | 1 h-2 h |
| | | | | | | | (end of | (end of | (end of |
| | | | | | | | distillation) | distillation) | distillation) |

| | | | | | |
|---|---|---|---|---|---|
| Final NOH(mg/KOH/g | 271.9 | 268.8 | 90.3 | 202.1 | 88.7 |
| reaction medium | | | | | |
| NFT 60 231) | | | | | |

2^{nd} step: transesterification of the reaction products by SOME

| n OH/n SOME | 1/1 | | 1/1 | | 1/1 | | 1/1 | | 1/1 | | 1/1 | |
|---|---|---|---|---|---|---|---|---|---|---|---|---|
| % of MeONa catalyst | 0.5% | | 0.5% | | 0.5% | | 0.72% | | 0.72% | | 0.72% | |
| (MeONa mass based on | m/mSOME | | m/mSOME | | m/mSOME | | m/mSOME | | m/mSOME | | m/mSOME | |
| the introduced mass of | | | | | | | | | | | | |
| SOME) | | | | | | | | | | | | |
| T° introduction of the | at 80° | C. | at 80° | C. | at 90° | C. | at 20° | C. | at 20° | C. | at 20° | C. |
| catalyst | | | | | | | | | | | | |
| % DMPS | 10 | ppm | 10 | ppm | 10 | ppm | 10 | ppm | 10 | ppm | 10 | ppm |
| Reaction temperature | from 90 to | | from 90 to | | from 90 to | | 20 to | | 20 to | | 20 to | |
| ° C. | 130° C. | | 130° C. | | 130° C. | | 165° C. | | 165° C. | | 165° C. | |
| Pressure mbars | 30 | mbars | 30 | mbars | 47 | mbars | 30 | mbars | 30 | mbars | 30 | mbars |

| | | | | | | | | | | | |
|---|---|---|---|---|---|---|---|---|---|---|---|
| Reaction time (h) | 7 | h | 7 | h | 2 | h | 4 | h | 6 | h | 3 h 30 |

| | | | | |
|---|---|---|---|---|
| Final OHN final (mg | 9.4 | 8.9 | 3.1 | 5.6 |
| KOH/g of reaction | | | | |
| medium, NF T 60-231) | | | | |

Neutralization of the OH residuals

| Nature of the added acid | Acetic acid | | Acetic | |
|---|---|---|---|---|
| | | | anhydride | |
| Nb of moles of acid/nb | 1/1 | | 1/1 | |
| of moles of free OH + | | | | |
| nb of MeONa introduced | | | | |
| in the 1^{st} and 2^{nd} step) | | | | |
| Reaction time (h) | 5 | h | 1 | h |
| Reaction temperature | 120° | C. | 140° | C. |
| (° C.) | | | | |

| | | | | | | |
|---|---|---|---|---|---|---|
| Final OHN (mg KOH/g | 37.0 | 3.3 | 9.4 | 8.9 | 3.1 | 5.6 |
| of reaction medium, | | | | | | |
| NF T 60-231) | | | | | | |

Characterization of the Samples:

The samples of PET esters prepared as described earlier, were characterized by the following methods:

1.1 Composition of fatty acid methyl esters (FAME): NF ISO 5509 (preparation of fatty acid methyl esters from samples), followed by NF ISO 5508 (GPC analysis of the prepared FAMEs).

NF ISO 5508 gives mass percentages of the different FAMEs present in the samples. From this mass composition, and knowing the molar masses of the different FAMEs, it is possible to calculate the molar percentages, n1 of short fatty acid methyl esters and n2 of long fatty acid methyl esters, respectively, based on the total number of FAME moles present in the sample.

The ratio between the number of moles of long fatty acids and the number of moles of short fatty acids is then calculated, characteristic of the oils according to the invention, n2/n1.

A “short” fatty acid methyl ester will be of formula RCOO CH₃, with R being an olefinic or paraffinic chain comprising from 6 to 11 carbon atoms (further designated by C₈-C₁₀).

A “long” fatty acid methyl ester will be of formula RCOO CH₃, with R being an olefinic or paraffinic chain comprising from 13 to 21 carbon atoms (further designated by C₁₈).

1.2: Ester composition by GPC: this is the determination of the mass percentages, based on the total sample weight, of the different categories of polyol esters (here PET) present.

The method used is a method by gas phase chromatography (GPC), which takes up again the characteristics of the IUPAC 2.323 method used for determining triglycerides.

The distinctive characteristics of the GPC method giving the PET ester composition of the oils according to the invention are specified below:

A short apolar column is used of the DB1 HT type (length: 15 m, internal diameter: 0.32 mm and thickness of the film: 0.1 μm).

The injector is of the on-column type and detection is by FID.

The separation is then only accomplished per increasing number of carbon atoms. In order to determine the retention time of the different esters, a mixture of triglycerides of known composition is passed therethrough as a reference, and the compounds with an equivalent number of carbon atoms are identified.

The reference used here is a mixture: AMF: referenced by the EEC, covering compounds with 24 to 56 carbon atoms.

Preliminary silylation is required in order to distinguish the partial esters from the total esters. In non-silylated samples, the presence of OH groups on the partial esters leads to smears upstream from the peaks. This smear disappears once the samples are silylated.

Therefore two measurements are carried out, respectively on non-silylated samples and silylated samples, and the respective amounts of partial and total esters are obtained by difference.

The silylation is accomplished under the following conditions: 10 mg of the sample are mixed with 200 μL of a BSTFA (bis trimethyl silyl trifluoracetamide)/TMSC1 (chlorotrimethyl silyl) (80/20 by volume) mixture. The whole is placed in the oven at 65° C. for 1 hour and vortexed from time to time. The sample is then diluted in iso-octane in order to obtain a concentration of 1 mg/mL.

The GPC Analysis Conditions are the Following:
50-370° C./min at 10° C./min, plateau of 10 min.
1 μL injection
1.2 bars of H₂

With this method it is possible to distinguish the polyol tetraesters (PET in the examples) including:

Four long chains (designated hereafter by 4C₁₈ in the examples) Three long chains and one short chain (designated hereafter by 3C₁₈1C₈ in the examples)

Two long chains and one short chain (designated hereafter by 2C₁₈2C₈ in the examples).

The “partial” esters which here comprise both esters having one or more non-esterified OH functions, tetraesters including three short chains and one long chain (3C₈1C₁₈ in the examples), tetraesters including four short chains (4C₈ in the examples). These three types of compounds cannot be separated from each other because of their too close number of carbon atoms.

The results are given as a mass percentage based on the total chromatographable species. The latter comprise:

The unreacted reaction products (polyol, C₇-C₁₂ short fatty acid methyl esters, C14-C22 long fatty acid methyl esters),

Partial esters (for all the products according to the invention, partial esters include tetraesters with three short chains and one long chain, as well as tetraesters with four short chains and esters having one or more non-esterified OH functions),

Tetraesters (other than those included in partial esters).

The retention times of the different species analyzed in the examples are detailed in Table 3 below). These retention times slightly vary according to the condition of the column. According to the IUPAC indications, one skilled in the art will know how to take these developments into account by recalibrating the column by passing again the reference through the latter.

Also, one skilled in the art will also know, for any product according to the invention and depending on the nature of the raw materials used (methyl esters and polyol) how to calibrate the column with an adequate mixture of reference triglycerides and identify the species per equivalent number of carbon atoms.

TABLE 3
GPC analysis retention time of the chromatographable species in the
samples

| Chromatograpable species | Retention time (min) |
|---|---|
| Short methyl esters (C₇H₁₅COOCH₃)) | 0.8 |
| Short methyl esters (C₁₀H₂₁COOCH₃) | 2.2 |
| Pentaerythritol | 5.0 |
| Long methyl esters (C₁₅H₃₁COOCH₃) | 8.0 |
| Long methyl esters (C₁₈H₃₇COOCH₃)) | From 9.5 to 10.5 |
| Partial esters | From 10.7 to 27.7 |
| Tetraester 2C₁₈2C₈ | From 28 to 29.9 |
| Tetraester 3C₁₈1C₈ | From 30.3 to 31.7 |
| Tetraester 4C₁₈ | From 32.9 to 35.6 |

The Measured Characteristics are:
The acid number (NF EN ISO 660), in mg of KOH/gram of product with which the unreacted fatty acids may be quantified (the higher the number, the more there are unreacted fatty acids
The iodine number (NF EN ISO 3961), in g of I₂/gram of product, which is related to the presence of unsaturation and therefore to the sensitivity to oxidation (the higher the number and the more there are unsaturations, the resistance to oxidation is therefore not as good;
The hydroxyl number (NF T 60-231), in mg of KOH/gram of product, with which the unreacted hydroxyl functions may be quantified (the higher the number and the more there are unreacted hydroxyl functions in the medium)
Kinematic viscosity at 40° C. (KV40) and at 100° C. (KV100), (ASTM D445), in mm²/s, calculation of VI (ASTM D2270)
Low temperature dynamic viscosity (CCS at −25° C., ASTM D5293), in mP.

The compositions and the physicochemical characteristics of the prepared oils are grouped in Table 4 below.

The oils PET 9-1, PET 12-1, PET 25-3, PET 28-2, and PET 29-1 are oils according to the invention. The oil PET 15-3 is not according to the invention.

TABLE 4
characteristics and properties of PET esters

| | | Sample | | | | | |
|---|---|---|---|---|---|---|---|
| | | | | PET 15-3 | | | |
| | | | | Non- | | | |
| | | PET 9-1 | PET 12-1 | inventive | PET 25-3 | PET 28-2 | PET 29-1 |
| Acid number | | 0.38 | 3.32 | 1.30 | 0.74 | 0.48 | 0.20 |
| (mg KOH/g) | | | | | | | |
| Iodine number | | n.d | n.d | 57.1 | 34.0 | 43.0 | 21.0 |
| (g I₂/100 g) | | | | | | | |
| Hydroxyl number | | 37.0 | 3.3 | 9.4 | 8.9 | 3.1 | 7.8 |
| (mg KOH/g) | | | | | | | |
| FAME | n1 C₈C₁₀ “short” | 35.0 | 38.0 | 49.4 | 61.4 | 50.1 | 70.4 |
| composition | n2 C₁₈ “long” | 65.0 | 62.0 | 47.1 | 38.6 | 49.9 | 29.6 |
| (molar %), | n2/n1 | 1.86 | 1.63 | 0.95 | 0.63 | 0.99 | 0.42 |
| according to | | | | | | | |
| NF ISO | | | | | | | |
| 5509/5508 | | | | | | | |
| Ester | Tetraesters (total) | 55.6 | 61.8 | 36.1 | 33.9 | 59.2 | 40.7 |
| composition | 2C₁₈ 2C₈ | 26.1 | 21.6 | 6.1 | 22.4 | 26.6 | 20.9 |
| (mass %) | 3C₁₈ 1C₈ | 20.4 | 30.6 | 15.9 | 9.7 | 22.8 | 14.2 |
| GPC | 4C₁₈ | 9.1 | 9.6 | 14.1 | 1.8 | 9.8 | 5.6 |
| | Partial esters | 37.7 | 34.2 | 60.5 | 57.8 | 39.2 | 49.2 |
| Comments | | OH | OH | 58.9% of | | | |
| | | neutralized | neutralized | free C₁₈ | | | |
| | | by acetic | by acetic | | | | |
| | | acid | anhydride | | | | |
| KV 100 mm2/s | | 8.6 | 8.4 | 2.7 | 7.3 | 9.2 | 6.2 |
| KV40 mm2/s | | 43.9 | 41.9 | 9.1 | 36.6 | 50.2 | 30.3 |
| VI | | 178 | 184 | 148 | 167 | 169 | 159 |
| CCS −25° C., | | 4250 | 3300 | n.d. | 9950 | 10600 | 10180 |
| mPa/s | | | | | | | |
| Storage stability | t = 0 | limpid | limpid | limpid | slightly | limpid | limpid |
| at 60° C. | | | | | cloudy | | |
| | 1 week | limpid | limpid | limpid | slightly | limpid | limpid |
| | | | | | cloudy | | |
| | 1 month | limpid, | limpid, | limpid color | slightly | limpid, | limpid, |
| | | color | color | unchanged | cloudy | color | color |
| | | unchanged | unchanged | | color | unchanged | unchanged |
| | | | | | unchanged | | |
| Storage stability | t = 0 | limpid | limpid | limpid | slightly | slightly | slightly |
| at 0° C. | | | | | cloudy | cloudy | cloudy |
| | 1 week | deposit | slight | slightly | slight | slightly | deposit |
| | | | flocculation | cloudy | flocculation | cloudy | |
| | 1 month | deposit | slight | slight | slight | slight | deposit |
| | | | flocculation | flocculation | flocculation | flocculation | |

The samples PET 9-1 and PET 12-1, which were subject to a step for neutralizing residual hydroxyl functions by acetic acid or acetic anhydride, have a viscosity compatible with a use as lubricating oils. However, they are a little viscous for an engine application: their viscosity at 100° C. is comprised between 8 and 9 cSt, while the mixtures of base oils in the formulations of the 5W30 type are stuck around 4 to 5 cSt. Their viscosity on the other hand is well adapted to the industrial lubricant application.

The cold properties (CCS to −25° C.) are good for the oils according to the invention while for the oil PET 15-3, these properties are so poor that they could not be measured.

The sample PET 15-3 has a viscosity of 2.7 cSt at 100° C., then becoming too low with respect to engine or industrial applications.

The samples PET 25-3, PET 28-2 and PET 29-1 are oils according to the invention. Their viscosity of 100° C. is closer to the target of 6 cSt and is suitable for an engine application.

Low temperature properties are seen to be not as good as those for the samples PET 9-1, 12-1 and 15-3: the CCS viscosities at −25° C. of PET 25-3, PET 28-2 and PET 29-1 are comparable with those of Group I mineral oils. However it is possible, considering these values, to formulate oils with a viscosimetric grade compliant with use for engines, by including in the formulation, suitable polymers and pour point additives (PPD).

It may be expected that these oils have low volatility, as in the case of rapeseed oil.

Stability:

Stability tests are carried out in a test tube in a weathering enclosure. Most samples are limpid and stable at room temperature and at 60° C. A tendency to deposit formation is observed after extended storage at 0° C., probably resulting from the presence of compounds or impurities having a high pour point. This point may be improved by better purification of the product.

### Example 2

### Thermo-Oxidative Properties of the Oils Based on PET Ester

The thermo-oxidative properties of the PET esters described in Example 1 were evaluated in a screening formulation consisting of 91.9% of said oils and 8.1% by weight of a package of additives having standard performance for engine oils, marketed by Lubrizol under reference 7819H. As a comparison, these screening formulations were also prepared from two widely available vegetable oils, oleic sunflower oil with 85% of oleic acid and rapeseed oil.

This evaluation is accomplished from laboratory ICOT and MCT tests.

ICOT Evaluation:

The ICOT (Iron Catalyzed Oxidation Test) is described in the ASTM D4871-06 standard (or ASTM D4871). It consists of bringing the lubricant to a temperature comprised between 50 to 375° C., in the presence of air, oxygen, nitrogen or another gas at a flow rate from 1.3 to 13L/h, with or without iron catalyst. The relative change in viscosity at 40° C., RKV40 (%) obtained after the ICOT test is then measured.

The tests were conducted here at 170° C., for 72 hours in the absence of iron.

The results are grouped in Table 5:

TABLE 5
increase in KV 40 (RKV40%) after the oxidation test ICOT.

| | RKV40% |
|---|---|
| PET 9-1 | More than 5,000 |
| PET 12-1 | More than 500 |
| PET 25-3 | 2400 |
| PET 29-1 | 950 |
| Refined grapeseed oil | More than 5,000 |
| 85% oleic sunflower oil | More than 5,000 |

Comparatively with standard vegetable oils, the oils 25-3 and 29-1 according to the invention have significantly improved resistance to oxidation, being expressed by a lesser increase in the viscosity at 40° C. after the ICOT test.

MCT evaluation:

The MCT (Micro Coking Test) is a test with which the tendency of forming deposits on a hot surface (coking) may be evaluated.

The MCT test evaluates the thermal stability of a thin film lubricant, subject to temperature conditions similar to those encountered in the hottest portions of the engine (230 to 280° C.). The deposits and varnishes are measured by a video grader. The result is expressed in the form of a score out of 10, called quality.

The test conditions are the following:
600 μL of oil (+10 ppm of anti-foam agent)
duration: 90 min
a 1-2% tilted plate including a trough
a temperature gradient from 230 to 280° C.
video grading of the varnishes of the plate method 2, a so-called “division of the squares (/10)” method: score from 0 to 10, best result 10.

In this evaluation, a comparison with mineral or synthetic bases well known to one skilled in the art PAO8 (Group IV) 330 NS (Group I), Priolube 1976 (mono-ester, group V), and Priolube 3985 (diester, group V) is also included.

The results of this grading are grouped in Table 6.

The aspect of the plates after the MCT tests with the oils 330 NS, PET 9-1, PET 29-1, shows the significant improvement obtained with PET 29-1.

TABLE 6
grading after the MCT test

| | MCT test quality/10, meth. 2 video |
|---|---|
| Samples | grading |
| PET 9-1 | 5.8 |
| PET 12-1 | 5.5 |
| PET 28-2 | 6.9 |
| PET 25-3 | 6.5 |
| PET 29-1 | 8.4 |
| Refined rapeseed oil | 6.5 |
| 85% oleic sunflower oil | 5.5 |
| PAO8 | 7.5 |
| 330 NS | 7.5 |
| Priolube 1976 | 7.2 |
| Priolube 3985 | 8.6 |

The mixed esters 9-1 and 12-1 have very poor behavior as compared with the mineral (33 NS) and synthetic (PAO8, Priolube 3985) bases. Their behavior is similar to that of vegetable oils, with a significant formation of deposits.

On the other hand, the mixed esters according to the invention 28-2, 25-3 and 29.1 exhibit good performances, or even in the case of oil 29-1, performances which are higher or equivalent to those of commercial mineral and synthetic bases.

### Example 3

### Lubricating Compositions for Four-Stroke Engines

Compositions and physicochemical characteristics The oils based on PET esters obtained in Example 1 were included in an amount of 20% in two lubricant composition formulations for a four-stroke engine.

In each of the two formulations, the oils based on mixed esters are evaluated by comparison with a commercial ester, Priolube 3970, and well-known vegetable oils, rapeseed oil and 85% oleic sunflower oil.

These oils based on esters are used here as lubricant bases, in combination with commercial bases from group IV (polyalphaolefins): PAO4 Durasyn (kinematic viscosity of 4 cSt at 100° C.), PAO6 Durasyn (kinematic viscosity of 6 cSt at 100° C.), and PAO 8 Durasyn (kinematic viscosity of 8 cSt at 100° C.). The amounts of these commercial bases are adjusted so as to formulate oils of grade 30 (compositions A-I) and of grade 20 (compositions J-P).

The compositions A-1, and J-P respectively, also differ by the nature of the additives used. The table below gives the characteristics of the additives of both formulations made.

TABLE 7
additivation of oil formulations for 4-stroke engines.

| | | |
|---|---|---|
| Trade name | Type of additive | Type of molecule |

Compositions A to I (Formulation G05162A) (Formulation 1)

| | | |
|---|---|---|
| IDN3276 | Friction modifier (FM) | |
| Irganox L57 | Antioxidant | |
| Viscoplex 1-256 | Pour point depressor (ppd) | |
| IDN3269F | Package (Infineum) for a 4- | |
| | stroke engine oil | |
| SV206 | Polymer improving VI (VII) | OCP |

Compositions J-P (Formulation G02300F) (Formulation 2)

| | | |
|---|---|---|
| XOA3041C | Package (Oronite) for a 4-stroke | |
| | engine oil | |
| SV261 | Polymer improving VI (VII) | OCP |
| PI156S | Pour point depressor (ppd) | |
| Irganox L57 | Antioxidant | |

The compositions and physicochemical properties of the different lubricating compositions obtained, as well as the results of ICOT and MCT tests are given in Table 8 and Table 9.

The compositions D, E, F, are lubricating compositions according to the invention, as well as the compositions K, L, M.

The compositions A, B, C, as well as composition J were made with oils based on mixed esters which are not oils according to the invention.

Compositions G, H, as well as compositions 0, P, were made with known vegetable oils, rapeseed oil and 85% oleic sunflower oil.

The compositions I and N were made with the commercial ester Priolube 3970.

ICOT Oxidation Tests (T° 170° C., Test Duration 72 Hours, 40 ppm Fe)

The lubricating compositions according to the invention additived according to formulation 1 better resist to oxidation than those additived according to formulation 2.

In any case, the lubricating compositions according to the invention, formulated from oils based on PET esters, exhibit a significantly improved behavior as compared with compositions formulated from standard vegetable bases (85% oleic sunflower oil and rapeseed oil). The composition F has a behavior which is closer to that of composition I, based on a commercial ester.

MCT Test: test conditions
600μL of oil (+10 ppm of anti-foam agent)
duration P 90 min
1-2% tilted plate including a trough
temperature gradient from 230 to 280° C.
video grading of the varnishes of the plate method 2, a so-called “division of the squares(/10)” method.

All the lubricating compositions made with oils based on mixed esters are positioned between the vegetable bases (rapeseed oils and oleic sunflower oil) and the commercial synthetic ester Priolube 3970.

In particular, the lubricating compositions additived according to formulation 2, have significantly higher performances than those of the vegetable bases. In both types of additivation, the lubricating compositions according to the invention, F and M are equivalent to the compositions prepared with the commercial ester Priolube 3970.

Viscosimetric Properties:

Lubricants of grade 30 (compositions D, E, F), and of grade 20 (compositions K, L, M), were able to be formulated from the oils of PET 28-2, PET 25-3, PET 29-1 according to the invention.

Considering the values of CCS at −35° C. of the compositions D, E, F, formulation of engine lubricants of grade 5W30 in the SAE classification is possible.

On the other hand, these CCS values at −35° C. do not observe the specifications required for engine lubricants of grade 0W30 according to the SAE classification, however it seems possible, considering these same values, to formulate such lubricants of grade 0W30 by adapting the additivation, notably as regards the nature of the polymer and of the pour point depressor additive (ppd).

TABLE 8
compositions and properties of four-stroke engine lubricants: formulation 1

| | Nature | A | B | C | D | E | F | G | H | I |
|---|---|---|---|---|---|---|---|---|---|---|
| Tested | | PET | PET | PET | PET | PET | PET | Refined | 85% | Priolube |
| ester | | 9-1 | 12-1 | 15-3 | 28-2 | 25-3 | 29-1 | rapeseed | oleic | 3970 |
| | | | | | | | | oil | sunflower | |
| | | | | | | | | | oil | |
| | | Composition (mass %) | | | | | | | | |
| Bases | | | | | | | | | | |
| Ester | | 20.0% | 20.0% | 20.0% | 20.0% | 20.0% | 20.0% | 20.0% | 20.0% | 20.0% |
| PAO 6 | | 4.7% | 4.7% | 29.5% | 0.0% | 10.5% | 24.0% | 6.0% | 4.7% | 31.3% |
| Durasyn | | | | | | | | | | |
| PAO 8 | | 55.9% | 55.9% | 31.0% | 60.5% | 50.0% | 36.5% | 54.5% | 55.9% | 29.2% |
| Durasyn | | | | | | | | | | |
| Additives | | | | | | | | | | |
| IDN3276 | MF | 1.0% | 1.0% | 1.0% | 1.0% | 1.0% | 1.0% | 1.0% | 1.0% | 1.0% |
| Irganox | Antiox. | 1.0% | 1.0% | 1.0% | 1.0% | 1.0% | 1.0% | 1.0% | 1.0% | 1.0% |
| L57 | | | | | | | | | | |
| Viscoplex | ppd | 0.1% | 0.1% | 0.1% | 0.1% | 0.1% | 0.1% | 0.1% | 0.1% | 0.1% |
| 1-256 | | | | | | | | | | |
| IDN3269F | Package | 9.7% | 9.7% | 9.7% | 9.7% | 9.7% | 9.7% | 9.7% | 9.7% | 9.7% |
| | (Infineum) | | | | | | | | | |
| SV206 | VII | 7.7% | 7.7% | 7.7% | 7.7% | 7.7% | 7.7% | 7.7% | 7.7% | 7.7% |

| | Physicochemical properties | | | | | | | | |
|---|---|---|---|---|---|---|---|---|---|
| KV 100 mm2/s, | 12.12 | 12.20 | 10.85 | 11.48 | 11.87 | 12.04 | 12.19 | 12.17 | 11.75 |
| ASTM D445 | | | | | | | | | |
| KV 40 mm2/s, | 65.11 | 65.14 | 56.27 | 62.01 | 64.91 | 66.41 | 65.44 | 64.79 | 65.34 |
| ASTM D445 | | | | | | | | | |
| VI, ASTM D2270 | 186 | 188 | 188 | 182 | 182 | 180 | 187 | 189 | 178 |
| CCS at −35° C., ASTM | 6144 | 7197 | | 6287 | 8251 | 9135 | 4860 | 4954 | 5368 |
| D5293 in mPa · s | | | | | | | | | |
| ICOT 72H 60 ppm Fe | 94/145 | 139 | 33/80 | 8/43 | 14 | −4 | Caking | 117 | −19.4/−19.5 |
| RKV 40(%) | | | | | | | | | |
| MCT quality/10 | 6.6 | 7.8 | 7.9 | 7.2 | 7.4 | 8.8 | 6.4 | 5.2 | 9.5 |
| (video grading | | | | | | | | | |
| method 2) | | | | | | | | | |
| MCT initial | 230 | 230 | 232 | 230 | 235 | 249 | 230 | 230 | 252 |
| temperature (° C.) | | | | | | | | | |

TABLE 9
composition and properties of four-stroke engine lubricants: formulation 2

| | Nature | J | K | L | M | N | O | P |
|---|---|---|---|---|---|---|---|---|
| Tested | | PET | PET | PET | PET | Priolube | Refined | 85% oleic |
| ester | | 9-1 | 28-2 | 25-3 | 29-1 | 3970 | rapeseed | sunflower |
| | | | | | | | oil | oil |
| | | Composition (mass %) | | | | | | |
| Bases | | | | | | | | |
| Ester | | 20.0% | 20.0% | 20.0% | 20.0% | 20.0% | 20% | 20.0% |
| PAO 6 | | 21.0% | 17.0% | 40.4% | 42.0% | 50.0% | 21.0% | 21.0% |
| Durasyn | | | | | | | | |
| PAO 4 | | 39.0% | 43.0% | 19.6% | 18.0% | 10.0% | 39.0% | 39.0% |
| Durasyn | | | | | | | | |
| Additives | | | | | | | | |
| XOA3041C | Package | 13.3% | 13.3% | 13.3% | 13.3% | 13.3% | 13.3% | 13.3% |
| | (Oronite) | | | | | | | |
| SV261 | VII | 4.4% | 4.4% | 4.4% | 4.4% | 4.4% | 4.4% | 4.4% |
| PI156S | ppd | 0.1% | 0.1% | 0.1% | 0.1% | 0.1% | 0.1% | 0.1% |
| Irganox | Antiox. | 0.2% | 0.2% | 0.2% | 0.2% | 0.2% | 0.2% | 0.2% |
| L57 | | | | | | | | |

| | Physico-chemical properties | | | | | | |
|---|---|---|---|---|---|---|---|
| KV 100 mm2/s, | 9.90 | 8.65 | 9.77 | 9.63 | 9.96 | 10.05 | 10.12 |
| ASTM D445 | | | | | | | |
| KV 40 mm2/s, ASTM | 54.16 | 46.30 | 54.91 | 53.45 | 55.18 | 53.12 | 53.70 |
| D445 | | | | | | | |
| VI, ASTM D2270 | 172 | 168 | 165 | 167 | 169 | 180 | 179 |
| ICOT 72H 60 ppm Fe | Caking | 73 | 179 | 185 | 28 | Caking | Caking |
| RKV 40(%) | | | | | | | |
| MCT quality/10 | 7.0 | 6.6 | 7.3 | 8.5 | 8.3 | 5.4 | 4.5 |
| (video grading | | | | | | | |
| method 2) | | | | | | | |
| MCT initial | 230 | 231 | 230 | 246 | 245 | 230 | 230 |
| temperature (° C.) | | | | | | | |

1. An oil comprising at least one tetraester fitting the general formula (I): 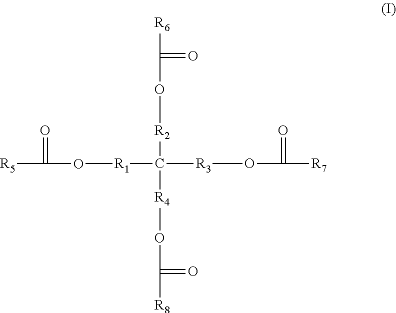
wherein:
the groups R₁, R₂, R₃, R₄ are aliphatic chains including from 1 to 10 carbon atoms
the groups R₅, R₆, R₇, R₈ are either short paraffinic chains including from 6 to 11 carbon atoms, or long olefinic chains including from 13 to 21 carbon atoms;
at least one of the groups R₅, R₆, R₇, R₈ is a short paraffinic chain including from 6 to 11 carbon atoms and at least one the groups R₅, R₆, R₇, or R₈ is a long olefinic chain including from 13 to 21 carbon atoms,
wherein,
the ratio between the number of moles of long fatty acids comprising from 14 to 22 carbon atoms and the number of moles of short fatty acids comprising from 7 to 12 carbon atoms, is comprised between 0.3 and 2.5, the ratio being determined on the composition of fatty methyl esters obtained from said oil by applying the NF ISO 5509 and NF ISO 5508 standards,
and wherein,
said oil comprises at least 15% by weight, preferentially at least 18% by weight, of tetraester(s) of formula (I) wherein 2 of the groups R₅, R₆, R₇, R₈ are short paraffinic chains including from 6 to 11 carbon atoms, and 2 of the groups R₅, R₆, R₇, R₈ are long olefinic chains including from 13 to 21 carbon atoms.

2. The oil according to claim 1 wherein R₁, R₂, R₃, R₄ are aliphatic chains including from 1 to 4 carbon atoms.

3. The oil according to claim 1, wherein the long fatty acid methyl esters comprising from 14 to 22 carbon atoms are in majority mono-unsaturated, in the composition of fatty acid methyl esters obtained from said oil by applying the NF ISO 5509 and NF ISO 5508 standards.

4. The oil according to claim 1 comprising at least 30% by weight, of tetraesters of formula (I), wherein at least two of the groups R₅, R₆, R₇, R₈ are long olefinic chains including from 13 to 21 carbon atoms and/or of a tetraester fitting the general formula (II) 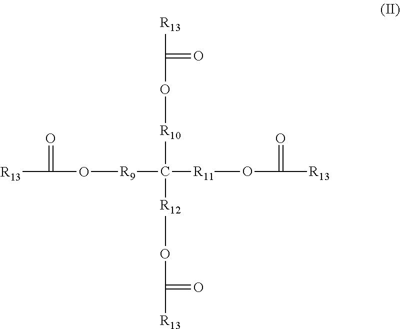
wherein R₉, R₁₀, R₁₁, R₁₂ are aliphatic chains including from 1 to 10 carbon atoms, preferentially from 1 to 4 carbon atoms, and R₁₃ is a long olefinic chain including from 13 to 21 carbon atoms.

5. The oil according to claim 4 comprising at most 10% by weight of tetraesters of formula (II).

6. The oil according to claim 1, comprising at most 25% by weight of tetraester of formula (I) wherein 3 of the groups R₅, R₆, R₇, R₈ are long olefinic chains including from 13 to 21 carbon atoms.

7. The oil according to claim 1 comprising at least 85% by weight of partial or total ester(s) obtained by reaction of one or more polyols of formula (III) 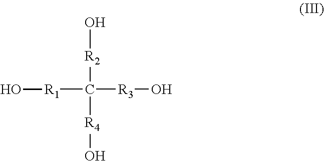
wherein R₁, R₂, R₃, R₄ are aliphatic chains including from 1 to 10 carbon atoms, preferentially from 1 to 4 carbon atoms, with one or more long unsaturated fatty acids comprising 14 to 22 carbon atoms and/or one or more short saturated fatty acids comprising 7 to 12 carbon atoms.

8. The oil according to claim 1 comprising at least 30% by weight of tetraesters of formula (I) including 40 to 70 carbon atoms and at least 15% by weight of tetraesters of formula (I) including 45 to 60 carbon atoms.

9. The oil according to claim 1, wherein the ratio between the number of moles of long fatty acids comprising from 14 to 22 carbon atoms and the number of moles of short fatty acids comprising from 7 to 12 carbon atoms, is comprised between 1.5 and 2.5, the ratio being determined on the composition of fatty acid methyl esters obtained from said oil by applying the NF ISO 5509 and NF ISO 5508 standards.

10. The oil according to claim 1, wherein the ratio between the number of moles of long fatty acids comprising from 14 to 22 carbon atoms and the number of moles of short fatty acids comprising from 7 to 12 carbon atoms, is between 0.4 and 1.1, the ratio being determined on the composition of fatty acid methyl esters obtained from said oil by applying the NF ISO 5509 and NF ISO 5508 standards.

11. A lubricating composition comprising an oil according to claim 1.

12. The lubricating composition according to claim 11 comprising from 10 to 99% of said oil.

13. The composition according to claim 11 further comprising:
from 0 to 70%, of one or more base oils selected from mineral oils of group III and/or synthetic oils of groups IV, V and VI,
from 0 to 30% one or more polymers improving VI,
from 0.2 to 10% of one or more antioxidant additives,
from 0.01 to 5% of one or more pour point depressor additives.

14. The composition according to claim 11, comprising from 30 to 70% of one or more base oils of the group IV, with a kinematic viscosity at 100° C. comprising 4 and 8 cSt.

15. The lubricating composition according to claim 11, the kinematic viscosity of which at 100° C. is between 5.6 and 9.3 cSt (grade 20).

16. The lubricating composition according to claim 11, the kinematic viscosity of which at 100° C. is between 9.3 and 12.5 cSt (grade 30).

17. The lubricating composition according to claim 11 having a viscosity index greater than 160.

18. An oil according to claim 1 wherein the oil is a friction modifier additive.

19. (canceled)

20. An oil according to claim 9 wherein the oil is a lubricating base, for a lubricant for hydraulics, transmissions and for industrial lubricants.

21. An oil according to claim 10 wherein the oil is a lubricant base for a lubricant for engines, hydraulics, transmissions and for industrial lubricants.

22. An oil according to claim 10 wherein the oil is a lubricant base for formulating a single lubricant which may be used both in engines, hydraulics and transmissions of public works' vehicles or farm vehicles.

23. A lubricating composition according to claim 1 as a lubricant for four-stroke engines.

24. A method for producing an oil according to claim 1 comprising:
i) a first step for transesterification of a polyol of formula (III): 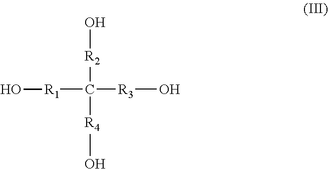
wherein the groups R₁ to R₄ are aliphatic chains including from 1 to 10 carbon atoms, by one or more saturated short fatty acid methyl esters including from 7 to 12 carbon atoms,
in the presence of a homogeneous or heterogeneous basic transesterification catalyst,
in an initial alcohol/saturated fatty acid methyl esters comprised between 1/5 and ½.5,
comprising the steps of:
i.1: introducing the catalyst at a temperature of the order of 20 to 25° C., into the reaction mixture formed by the polyol and the saturated short fatty acid methyl ester(s),
i.2. raising the temperature of the reaction mixture up to a temperature above 150° C.,
followed by
(ii) a second step for transesterification of one or more reaction products obtained in the first step (i), by one or more long unsaturated fatty acid methyl esters comprising from 14 to 22 carbon atoms,
in the presence of a homogeneous or heterogeneous basic transesterification catalyst,
comprising the steps of:
ii.1: measuring, according to the NF T 60-231 standard, the hydroxyl number of the starting medium formed by a determined amount of one or more products from a first step (i), and calculating the number of moles of non-esterified polyol hydroxyls, nOH, present in said medium,
ii.2: introducing into said medium at a temperature of the order of 20 to 25° C., N moles of long unsaturated fatty acid methyl ester(s) in a N/nOH molar ratio comprised between 0.8 and 1.2,
ii.3: introducing into said medium at a temperature of the order of 20 to 25° C., an amount of the catalyst representing between 0.5 and 1.5% by mass of the amount of long saturated fatty acid methyl esters introduced in step ii.2.

25. The method according to claim 24 further comprising a third step for neutralization of the unreacted hydroxyl groups by acetic anhydride.

26. The method according to claim 24, wherein the mixture of unsaturated long fatty acid methyl esters comprising from 14 to 22 carbon atoms used in step (i) for transesterifying the polyol, includes at least 85% by weight of mono-unsaturated fatty acid methyl esters, said percentage being determined from NF IS05508.

27. The method according to claim 26, wherein the mono-unsaturated methyl esters comprise from 16 to 22 carbon atoms, carbon atoms.

28. The method according to claim 24, wherein the polyols are selected from pentaerythritol and neopentylglycol.
